(19) United States
(12) Patent Application Publication
(10) Pub. No.: US 2019/0022061 A1
(43) Pub. Date: Jan. 24, 2019

(54) Statins (Atorvastatin) can lower blood sugar level in diabetic

(71) Applicant: Kieu Hoang, Westlake Village, CA (US)

(72) Inventor: Kieu Hoang, Westlake Village, CA (US)

(21) Appl. No.: 16/042,826

(22) Filed: Jul. 23, 2018

Related U.S. Application Data
(60) Provisional application No. 62/535,276, filed on Jul. 21, 2017.

(51) Int. Cl. A61K 31/40; A61P 3/10

(52) U.S. Cl. CPC A61K 31/40; A61P 3/10

(57) ABSTRACT

A method of using Kieu Hoang wine gold label to lower down the blood sugar in the diabetics. Atorvastatin is used as a positive control to lower down blood sugar level.

This application claims the benefit of U.S. Provisional Patent Application 62/535,276, filed Jul. 22, 2017, which is incorporated herein by reference.

### BACKGROUND OF THE INVENTION

In our animal study using Kieu Hoang wine gold label can lower down the blood sugar in the diabetics. At the same time, we use Atorvastatin as a positive control and it is amazing that we also found Atorvastatin can also lower down blood sugar level.

### BRIEF DESCRIPTION OF THE DRAWINGS

FIG. 1A is a graph of blood glucose levels and AOC at zero to eight weeks.

FIG. 1B is a graph of blood glucose levels at 10-12 weeks.

FIG. 2A is a graph of insulin levels and AOC at zero to eight weeks.

FIG. 2B is a graph of insulin levels at 10-12 weeks.

FIG. 3A is a graph of OGTT Glucose AUC (0-120 min) post dose 63 days.

FIG. 3B is a graph of OGTT Glucose AUC (0-120 min) post dose 91 days.

### SUMMARY OF THE INVENTION

In our animal study using Kieu Hoang wine gold label can lower down the blood sugar in the diabetics. At the same time, we use Atorvastatin as a positive control and it is amazing that we also found Atorvastatin can also lower down blood sugar level.

### DETAIL DESCRIPTION OF THE INVENTION

1. KHGD Wine Control the Blood Glucose in an Obesity Animal Mode

(1) Executive Summary

The purpose of this study is to evaluate the effects of RAASUS products (KH103, KHJ and KHGD) in Diet-induced obesity (DIO) mice.

In this study, RAASUS products (KH103, KHJ and KHGD) were dosed three times a day (T ID) for 99 days. Atorvastatin was used as a positive control at a dose of 20 mg/kg three times a day (T ID). On day 63 post dosing, OGTT test were conducted for all of the mice. On the day 65 post dosing, 6 mice from each group were killed for bio-marker samples collection. Body weight and food intake were detected twice per week. On day −5, day 14, day 28 the non-fast blood was collected to determine the non-fast BG, insulin, TG, HDL-C, LDL-C and TCHO level. On day 42, day 56, day 70, day 84 the fast blood was collected to determine the fast BG, insulin, TG, HDL-C, LDL-C and TCHO level.

In this 99 days study, the data showed that:
1. The KHGD significantly decreased the blood glucose level and significantly increased the insulin sensibility.
2. All of the three products had no effects on liver TG and TCHO levels 8 weeks post dose.
3. The KHJ and KHGD significantly decreased the OGTT Glucose AUC(O-120 min) post dose 63 days; The KHGD significantly decreased the OGTT Glucose AUC(O-120 min) post dose 91 days.
4. Body weights and food intake in all compound treated groups have no significant difference compared with the vehicle treated group during the study.

(2) Study Summary

The study was initiated on Jul. 8, 2015 and completed on Sep. 22, 2015.

Study Purpose

The purpose of this study is to evaluate the effects of RAASUS compounds (KH103, KHJ and KHGD) in Diet-induced obesity (DIO) mice.

Study Results

In this 99 days study, the data showed that: 1. The KHGD significantly decreased the blood glucose level and significantly increased the insulin sensibility. 2. The KHJ and KHGD significantly decreased the OGTT Glucose AUC(O-120 min) post dose 63 days; The KHGD significantly decreased the OGTT Glucose AUC(O-120 min) post dose 91 days. 3. Body weights and food intake in all compound treated groups have no significant difference compared with the vehicle treated group during the study.

Materials and Methods

Experimental groups

| | | | Dose | Route | |
|---|---|---|---|---|---|
| | | Dose | Volume | of | |
| Group | Treatment | (mg/kg) | (mL/kg) | Admin | |
| 1 | Vehicle | | 10 | po | 10 |
| 2 | Atorvastatin | 20 | 10 | po | 10 |
| 3 | 1<1-1103 | | 10 | po | 10 |
| 4 | KHJ | | 10 | po | 10 |
| 5 | KHGD | | 10 | po | 10 |

Test System

Test article

| | | | | |
|---|---|---|---|---|
| Name: | Atorvastatin | KH 103 | KHJ | KHGD |
| Supplier: | Sigma | Rare Antibody | Rare Antibody | Rare |
| | | Antigen Supply | Antigen | Antibody |
| | | Inc. | Supply Inc. | Antigen |
| | | | | Supply |
| | | | | Inc. |
| Vehicle: | 0.5% HPMC + | dd water | dd water | dd water |
| | 2% Tween80 | | | |
| Lot: | LRAA2486 | | | |
| pMW | 1209.4 | | | |
| vMW | 1209.4 | | | |
| Purity | 100% | | | |
| Physical | White | Clear solution | Clear solution | Wine red |
| State: | powder | | | solution |
| Storage | | | | |
| Conditions | | | | |

Dose Formulation

The Atorvastatin formulation was prepared once a week and the KH103 formulation was prepared twice a week.

For Atorvastatin 20 mg/kg, dissolved 200 mg Atorvastatin in 100 ml 0.5% HPMC+2% Tween80, sonicated and votexed it until well dispersed.

For KH103, dissolved 1500 mg Atorvastatin in 10 ml dd water, sonicated and votexed it until well dispersed.

For KHJ and KHGD, they were provided by the Rare antibody antigene supply Inc.

These compounds were administered to animals in a volume of 10 mL/kg by oral gavage. Dosing solutions were prepared as needed in amber glass bottles and stored in refrigerator. Dose volumes were adjusted daily according to body weight.

Animal

| | |
|---|---|
| Species | DIO C57BL/6J mice |
| Justification for Species | Diet-induced obesity (DIO) in the C57BL/6J |
| Selection | mice was employed to evaluate the effect of |
| | anti-hyperlipidemia, anti-diabetics and |
| | increasing of insulin sensitivity effect in |
| | this study. |
| Body Weight Range | 40-50 g for DIO mice |
| Study start Age | 26 weeks old |
| Sex | |
| Source | Shanghai SLAC Laboratory Animal Co. LTD. |
| Address of Supplier | Songjiang, Shanghai, P.R. China |
| Method of Identification | The mice were singly housed per cage with a |
| | unique cage number. |
| Number of Animals for | 50 mice |
| Dosing | |

DIO Model

Male C57BL/6J mice (Shanghai Laboratory Animal Center, Shanghai, China, 5 weeks) were fed with high fat diet (D12492i, Research Diet, New Brunswick, N.J.) (caloric contribution: protein, 20%; fat, 60%; carbohydrates, 20%) ad lib for 21 weeks for the induction of obesity.

Treatment

The mice of group 1 and group 2 were orally dosed with vehicle or Atorvastatin-20 mg/l<g respectively three times per day at 9:00, 13:00, 17:00 and fresh water, ad libitum from Day O to Day 99.

The mice of Group 3, Group 4 were orally dosed with KH103, KHJ respectively three times per day at 9:00, 13:00, 17:00 and free drinl<KH103, KHJ respectively at day and night from Day O to Day 99.

The mice of Group 5 were orally dosed with KHGD three times per day at 9:00, 13:00, 17:00 and free drink KHGD at day and fresh water, ad libitum at night from Day O to Day 99.

Note: the mice of KHJ and KHGD treatment groups are pre treated with original formulation for 14 days and followed 22 days treatment with a new formulation, then in the next 77 days the KHJ; KHGD were changed to the old formulation again.

In Life Data Collection

Body weight and food intake (over 24 h) were recorded twice per week during diet induction phase and daily during the treatment period.

The random blood glucose, insulin level were measured using tailing nick once per two weeks on day −5, day 14, day 28.

The fast blood glucose (fast from 17:30 to 8:00 AM) insulin level were measured using tailing nick once per two weeks on day 42, day 56, day 70, day 84. The OGTT (oral glucose tolerance test) were carried out on day 63 and day 91.

Terminal Procedures

On day 65, 30 mice (5 groups*6 mice of each groups) were euthanized via C02 inhalation, liver was dissected, weighed and quicl<ly frozen on dry ice and stored at −80° C. for TG/TCHO detection. And the aortic arch was dissected, quicl<ly invaded into the RNAlater for inflammatory factor RNA detection.

On day 99, the rest 20 mice (5 groups*4 mice of each groups) were euthanized via C02 inhalation, liver were dissected and fixed by 4% paraformaldehyde for HE stain. And the spleen, draining lymph nodes and whole blood were collected for immunological analysis.

Sample Analysis

Blood Glucose: Blood glucose was determined using the Glucosemeter (Johnson and Johnson) and the Glucose Test Strips (Johnson and Johnson).

Plasma Insulin assay: Plasma insulin was determined using a commercial kit (Catalog #: EZRM1-13K) purchased from Millipore (Billerica, Mass., USA).

(3) Results

Biochemical Analysis:

Blood Glucose:

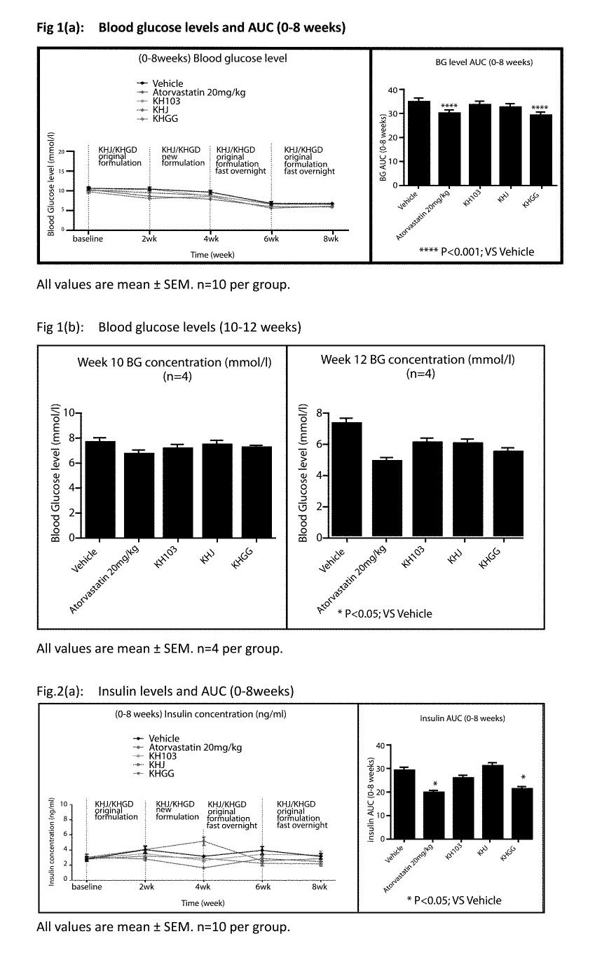

The Atorvastatin-20 mg/kg as positive control significantly decrease the blood glucose level AUC (0-8 weeks) compared with the vehicle groups. The KHGD significantly decrease the blood glucose level AUC (0-8 weeks) compared with the vehicle groups. The KH103 and KHJ have no effect on the blood glucose level AUC (0-8 weel<s) 8 weel<s post dose. (FIG. 1(a))

The Atorvastatin-20 mg/l<g as positive control significantly decrease the blood glucose level compared with the vehicle groups 12 weeks post dose. All the test articles have no effect on the blood glucose level 10 weeks and 12 weeks post dose due to the less animal numbers. (FIG. 1(b))

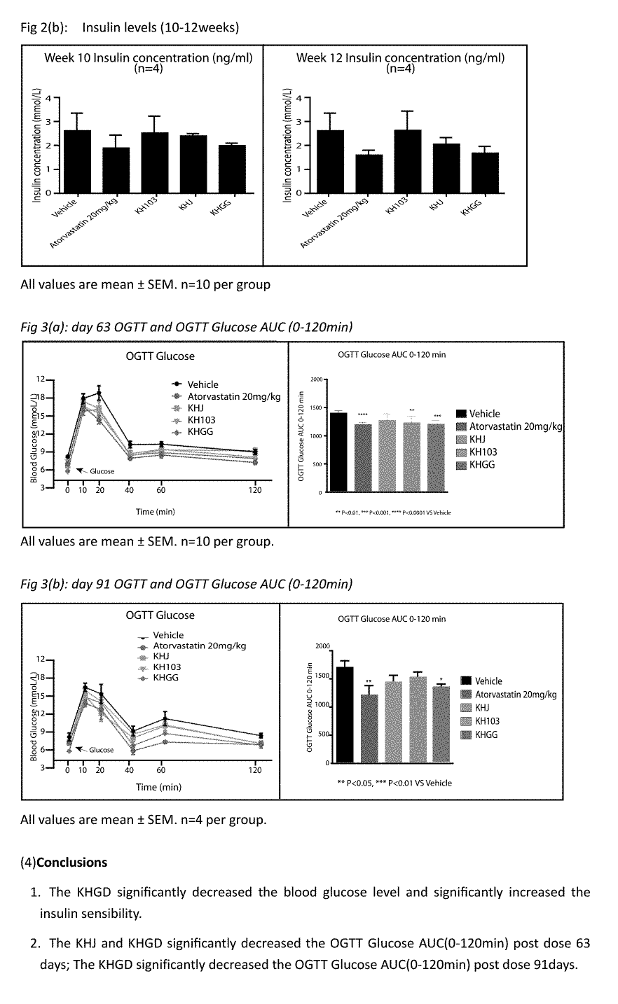

The Atorvastatin-20 mg/kg as positive control significantly decrease the insulin AUC (0-8 weeks) compared with the vehicle groups. The KHGD significantly decrease the insulin AUC (0-8 weeks) compared with the vehicle groups. The KH103 and KHJ have no effect on the insulin AUC (0-8 weel<s) post dose 8 weel<s this study. (FIG. 2(a))

The Atorvastatin-20 mg/kg and the KHJ, KHGD have a trend to decrease the plasma insulin level 10 weeks and 12 weeks post dose. (FIG. 2(b)) OGTT:

On the day 63, the Atorvastatin-20 mg/kg as positive control significantly decrease the OGTT glucose AUC (0-120 min) compared with the vehicle groups.

The KHJ and KHGD significantly decrease the OGTT glucose AUC (0-120 min) compared with the vehicle groups. The KH103 have no effect on the OGTT glucose AUC (0-120 min) post dose 63 days in this study. (FIG. 3(a))

On the day 91, the Atorvastatin-20 mg/kg as positive control significantly decrease the OGTT glucose AUC (0-120 min) compared with the vehicle groups.

The KHGD significantly decrease the

OGTT glucose AUC (0-120 min) compared with the vehicle groups. The KH103 and KHJ have no effect on the OGTT glucose AUC (0-120 min) 91 days post dose in this study. (FIG. 3(b))
1. The KGHD significantly reduced the blood glucose level and significantly increased the insulin sensibility.
2. 2. The KHJ and KGHD significantly decrease the OGTT Glucose AUC (0-120 min) post dose 63 days; The KGHD significantly decreased the OGTT Glucose AUC (0-120 min) post dose 91 days.
3. Body weights and food intake in all compound treated groups have no significant difference compared with the vehicle treated group during the study.

I claim:

1. A method of treating diabetes in a human in need thereof consisting essentially of administering a therapeutically effective amount of a statins like Atorvastatin to lower down the blood sugar level in diabetics

2. A method of curing diabetes in a human in need thereof consisting essentially of administering a therapeutically effective amount of a statins like Atorvastatin can be used to cure diabetics.
